(19) United States
(12) Patent Application Publication
(10) Pub. No.: US 2003/0017221 A1
(43) Pub. Date: Jan. 23, 2003

(54) Enzymatically catalyzed hydrolysis of corn fiber and products obtained from enzymatically hydrolyzed corn fiber

(75) Inventor: Richard L. Antrim, Solon, IA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Grain Processing Corporation, Muscatine, IA

(21) Appl. No.: 10/109,210

(22) Filed: Mar. 28, 2002

Related U.S. Application Data
(60) Provisional application No. 60/279,377, filed on Mar. 28, 2001.
(60) Provisional application No. 60/283,421, filed on Apr. 12, 2001.

(51) Int. Cl. A61K 35/78

(52) U.S. Cl. 424/750; 435/101

(57) ABSTRACT

Disclosed is a method for treating a grain fiber, in particular corn fiber. In accordance with the disclosed subject matter, the fiber is subjected to alkaline hydrolysis in the presence of a hemicellulose ferulate esterase in an aqueous medium. The hemicellulose ferulate esterase is present in an amount effective to catalyze alkaline hydrolysis, and the alkaline hydrolysis is performed under conditions suitable to permit the hemicellulose ferulate esterase to catalyze alkaline hydrolysis. The initial pH of the aqueous medium is alkaline but is sufficiently low to permit the enzyme to have catalytic activity. Preferably, the pH is in the range from 8.0 to 9.5. From the product mixture thus formed, fiber oil, cellulose, hemicellulose, and other materials may be obtained.

### CROSS-REFERENCE TO RELATED PATENT APPLICATIONS

This application claims priority to prior U.S. provisional application Serial No. 60/279,377, filed Mar. 28, 2001. This application also claims priority to prior U.S. provisional application Serial No. 60/238,421, filed Apr. 12, 2001. Both prior applications are hereby incorporated by reference in their entities.

### TECHNICAL FIELD OF THE INVENTION

The invention is in the field of grain fiber processing, in particular corn fiber processing. In preferred embodiments, the invention is directed towards the enzymatically catalyzed alkaline hydrolysis of corn fiber.

### BACKGROUND OF THE INVENTION

Industrial corn fiber is composed of a mixture of corn hulls (the pericarp of the corn kernel), starch, protein, germ fiber oil, and ash. The corn hull component (60% to 75% of the mixture) is composed of about 23% cellulose, 55% hemicellulose and 22% phenolic compounds, acetate and protein. The corn hull components are covalently bound together by ester and perhaps by other alkali-labile bonds to form a water-insoluble matrix. The hemicellulose molecule, which is water soluble in the free, non-esterified form, is composed mostly of an hydro 5 carbon sugars (55% xylose, 36% arabinose, and 7% galactose). The cellulose component in pure form is composed entirely of anhydro glucose. It is believed that hemicellulose and cellulose are covalently bonded through difunctional species, which act as cross-linking agents. The difunctional species are believed to consist largely of diferulates, with some dicoumerates also being present.

Corn fiber has numerous commercial uses. Principally, corn fiber is used as an animal feed, but corn fiber also can be used as a starting material in processes for obtaining cellulose and hemicellulose, which are of commercial value. Recently, it has been found that corn fiber contains an oil, known as “fiber oil” or “corn fiber oil,” that has nutritive properties and that may help lower serum cholesterol. The corn fiber oil is believed to be composed at least in part of esters that consist of cholesterol-like lipids, namely phytosterol. Corn fiber also contains cellulose. Cellulose in pure form, sometimes called “chemical grade” cellulose or “food grade” cellulose, is a material of commercial importance and is used in the manufacture of such products as chemically modified cellulosics (e.g. carboxymethylcellulose) and cellophane.

Conventionally, corn fiber is resolved into hemicellulose and cellulose by treating the fiber with high levels of caustic (NaOH) to thereby hydrolyze the crosslinking esters. Starch and at least part of the protein (that part which is not associated with the corn hulls) may be separated prior to the caustic treatment by treating the fiber with enzymes amylases and proteases, followed by filtration. The enzymes are believed to catalyze the hydrolysis of the glycoside and peptide bonds (the term “enzymatic hydrolysis” is sometimes used as shorthand for “enzymatically catalyzed hydrolysis”). For instance, one recently published document, U.S. Pat. No. 6,352,845 B1, issued Mar. 5, 2002 and assigned to Eastman Chemical Company, Kingsport, Tenn., purports to disclose the hydrolysis of protein catalyzed with a protease enzyme as a pretreatment preceding strong alkaline hydrolysis of corn fiber. The cellulose-rich component can be separated from the soluble mixture of ash, hemicellulose and phenolics by known methods, and hemicellulose can be separated from the phenolics by alcohol precipitation of the hemicellulose or by ultrafiltration.

Conventional processes suffer from numerous drawbacks. These processes require at least about 12% sodium hydroxide by dry weight, and in some cases substantially higher percentages. The conventional processes must be performed at high temperatures, which generally range from 100° to 120° C. Moreover, it is difficult to obtain fiber oil from corn fiber under the conventional processes. Although in theory fiber oil may be extruded with hexane or other organic solvents prior to strong alkaline hydrolysis of the corn fiber, this process is expensive and solvent-intensive and not commercially practicable. If the oil is not so extruded, the high temperatures and the harsh conditions involved in caustic treatment will destroy the oil. Finally, the cellulose-rich component obtained using the conventional processes is not pure cellulose, but rather is generally believed to be a covalently linked combination of hemicellulose and cellulose.

Given these drawbacks, it is often not commercially practicable to resolve corn fiber into products such as hemicellulose or its component sugars, and it is impractical to obtain fiber oil in large quantities. It is further difficult to obtain chemical grade cellulose from corn fiber. In one or more embodiments, the invention is addressed toward overcoming these drawbacks.

### THE INVENTION

The invention provides multiple products and methods in the field of grain fiber processing, in particular corn fiber processing. In accordance with a first embodiment of the invention, corn fiber is hydrolyzed under alkaline conditions, wherein the hydrolysis is catalyzed with a hemicellulose ferulate esterase. The hemicellulose ferulate esterase is an enzyme that comprises one or more active domains (i.e., active sites) which comprise one or more amino acids that catalyze the cleavage of one or more ferulic acid ester bonds within the corn fiber. The hydrolysis is performed under conditions suitable to permit the hemicellulose ferulate esterase to catalyze the alkaline hydrolysis. Specifically, the initial pH of the aqueous medium is alkaline, but is sufficiently low to permit the enzyme to have catalytic activity. In general, the reaction will consume caustic and the pH should be periodically or continuously adjusted by adding more caustic reagent to the reaction mixture. Preferably, the hydrolysis is performed at an optimal temperature for the enzyme and at a pH that is maintained in the range of about 8.0 to 9.5. Optionally, but preferably, a coumeric acid esterase is further employed to catalyze the hydrolysis. The coumeric acid esterase catalyzes the cleavage of one or more coumeric acid ester bonds with corn fiber. Preferably, an acetate esterase (acetate esterase) is further employed. It is believed that acetic acid is bonded via ester bonds to —OH groups on the hemicellulose and cellulose backbones. In the preparation of chemical grade cellulose, such acetic acid groups are undesired. Most preferably, an alkaline stable amylase and an alkaline stable protease are used concurrently with the foregoing esterases to hereby hydrolyze respectively polymeric starch and protein into smaller molecules.

In the most preferred embodiment, after the hydrolysis has been allowed to proceed to the desired termination point, a product mixture will be formed, the product mixture including fiber oil, cellulose, hemicellulose, low-DP sugars and other hydrolyzed starch byproducts, peptides, acetic acid, phenolic acids such as ferulic acid, and other materials. Commercially valuable products such as corn fiber oil, cellulose, hemicellulose, and phenolic acids may be recovered from this product mixture.

In a second embodiment of the invention, the corn fiber is hydrolyzed with coumeric acid esterase, with the use of hemicellulose ferulate esterase being optional. In a third embodiment, the corn fiber is hydrolyzed with acetate esterase, with the use of other esterases being optional.

In a fourth embodiment of the invention, corn fiber is hydrolyzed in an aqueous medium an initial pH less than 10.5, preferably less than 10.0, and more preferably in a range from 8.0 to 9.5. The use of catalyzing enzymes is optional in this fourth embodiment.

In a fifth embodiment of the invention, a method for obtaining a mixture of arabinose and xylan is provided. The method comprises providing an aqueous solution of hemicellulose, preferably hemicellulose recovered from a product mixture obtained as described hereinabove, and treating the hemicellulose in the aqueous solution with an arabinose-releasing enzyme under reactions conditions suitable to yield the mixture of arabinose and xylan. It is contemplated that arabinofuranosidases known in the art may be useful in conjunction with this embodiment of the invention. The xylan is an anhydro xylose-enriched polymer, which may be recovered from the mixture thus formed, and hydrolyzed, preferably via mild acid or enzyme hydrolysis (this term is used in this instance to refer to enzymatically catalyzed acid hydrolysis), to yield xylose.

The invention also encompasses products prepared in accordance with the methods described herein above, and also products obtained from a product mixture prepared via the hydrolysis of grain fiber as discussed hereinabove. Other features of preferred embodiments of the invention are described hereinbelow.

### DETAILED DESCRIPTION OF THE PREFERRED EMBODIMENTS

“Grain fiber” as contemplated herein is the hemicellulose-containing part of a grain, and may include, for instance, hulls, pericarp, or germ. The invention is contemplated to be applicable to any grain fiber that contains cellulose and hemicellulose, and is particularly applicable to wheat bran, oat hulls, and corn fiber. Most preferably, the fiber is corn fiber. Corn fiber is typically obtained from the corn wet or dry milling industries, and the corn fiber used in conjunction with the invention may be obtained via wet or dry milling or may be obtained commercially. The preferred embodiment will be discussed herein with respect to corn fiber hydrolysis, but it should be understood that the invention is applicable to the hydrolyses of other grain fibers.

The methods of the present invention preferably are performed with various catalyzing enzymes. In the highly preferred embodiment of the invention, a hemicellulose ferulate esterase is employed. Hemicellulose ferulate esterases, which contain one or more domains that catalyze the cleavage of ferulic acid esters, are a class of enzymes known in the art, and the hemicellulose ferulate esterase used in conjunction with the invention may be derived from a microbe using conventional techniques. For instance, it is known to isolate ferulic acid esterases from Aspergillus niger. Preferably, the microbe is obtained from an ecological niche associated with conditions under which cellulosic materials such as cellulose and hemicellulose are decomposed. It is believed that certain ferulic acid esterases will be specific to catalysis of the hydrolysis of hemicellulose, as opposed to esters that compose the corn fiber oil. The invention further contemplates the use of a coumeric acid esterase, which is a known class of enzymes that contains one or more domains that catalyze the cleavage of coumeric acid esters. Coumeric acid esterase may be isolated from microbes (e.g. the Aspergillus family) in accordance with known techniques. The hemicellulose ferulate esterase and coumeric acid esterase may be considered together as “hemicellulose liberating enzymes.”

Another class of enzymes useful in conjunction with the invention are acetate esterases, which may be selected from among known enzymes that contain one or more domains that catalyze the cleavage of acidic acid esters. Such enzymes may be microbially derived in accordance with conventional techniques, e.g. from the Fibrobacter family. Alkaline-stable protease and amylases may be employed in conjunction with the invention respectively to catalyze the hydrolysis of proteins and starches present in the corn fiber. A wide variety of proteases and amylases are known and available commercially from companies such as NOVO and GENENCOR, and alkaline stable proteases and possibly alkaline stable amylases are available commercially, or such enzymes may be derived microbially via known techniques. Generally, amylases and proteases may be derived from bacteria of the family Bacillus.

Arabinose-releasing enzymes, such as known arabinofuranosidases, further may be employed in conjunction with certain embodiments of the invention. The proteases, amylases and arabinofuranosidases may be microbially derived in accordance with known techniques, e.g. from the Bacillus family or a fungal family.

Finally, a xylanase enzyme (many of which are commercially available) may be used in certain embodiments of the invention to catalyze the hydrolysis of xylan. Xylanases, which are typically derived from the Tricoderma or Aspergillus families, are available from NOVO and GENENCOR. The forgoing discussion of enzymes is not meant to be exclusive of other enzymes, and indeed to the contrary other enzymes as may be known or hereinafter discovered may be used in conjunction with the invention. Likewise, combinations of two or more enzymes in a given class of enzymes (for instance, two or more amylases) may be used in a single catalysis.

The corn fiber may be hydrolyzed to form a product mixture at a low initial alkaline pH, preferably a pH in the range from 7.5 to 11, more preferably a pH less than 10.5, even more preferably a pH of less than 10.0, and most preferably a range from about 8.5 to 9.5. During the course of the hydrolysis, the pH preferably is maintained at a level that is at or near the initial pH, for instance, by employing a continuous bleed of caustic or by adding caustic at periodic intervals. When an enzyme is employed in conjunction with the hydrolysis, the initial pH should be a pH at which the enzyme is stable and effective for its intended purpose.

The corn fiber, water, and enzyme may be present with respect to each other in any suitable amounts. For instance, corn fiber may be hydrolyzed in an aqueous mixture in which the corn fiber is present in an amount of less than about 25% by weight, preferably about 10% by weight. It is contemplated that in an alternative embodiment an extrudable mixture of corn fiber and water will be provided, whereby the hydrolysis of the corn fiber may be allowed to proceed in an extruder. In either embodiment, each catalyzing enzyme should be present in any amount effective for its intended purpose. It will be appreciated that the selection of the reaction conditions and amount for the enzymatic hydrolyses disclosed herein will depend upon the specific characteristic of the enzyme and may vary not only among different suppliers but also from lot to lot within the same supplier. Thus, is it desirable to assess the optimal reaction conditions and the amounts of enzyme employed upon receipt of each new supply of enzyme. If the enzyme is obtained commercially, the supplier may provide a useful measurement of enzymatic activity, and this measure may aid in the determination of the appropriate reaction conditions. While a wide range of enzyme amounts may be employed, it is believed that the amount of enzyme will generally fall within the range from 0.01-0.1% by dry weight of the corn fiber.

The hydrolysis of corn fiber should be allowed to proceed until the desired termination point, which preferably is the point at which the hydrolysis of esters which crosslink hemicellulose and cellulose is complete or is as complete as is economically practical. For instance, the hydrolysis may be completed to 25%, 50%, 75%, 80%, 85%, 90%, 95%, or essentially 100%. The hydrolysis should proceed at a temperature in the range of 60°-90° C., preferably or in the range of 65-85° C., and most preferably about 70° C. Under these conditions, it is anticipated that the desired reaction time will be in the range from 2-12 hours, depending on enzyme dosage and other factors. The product mixture thus formed will include cellulose, hemicellulose, and corn fiber oil (in preferred embodiments). In preferred embodiments, the corn fiber is pretreated or treated concurrently with an amylase or protease enzyme, and in highly preferred embodiments, the fiber is pretreated or treated concurrently with both an amylase and a protease enzyme. In such event, the product mixture will further include low-DP sugars, oligsaccharides, peptides, acidic acid, phenolic acids (such as ferulic acid), and other materials. In other embodiments, the product mixture formed upon enzymatically catalyzed hydrolysis of ester bonds may be post-treated with an amylase enzyme, a protease enzyme, or both.

Preferably, the hydrolysis is performed to yield this product mixture, and other products subsequently are obtained from this product mixture, although is contemplated that in some embodiments a product mixture thus obtained may be supplied and used as a starting material.

From a product mixture thus prepared, it is contemplated that corn fiber oil may be recovered (although in some embodiments corn fiber oil may become hydrolyzed during the hydrolysis of the corn fiber). Preferably, when recoverable, the corn fiber oil is recovered via a mechanical treatment, such as skimming or centrifugation with, for instance, a three-phase centrifuge. Cellulose may be separated from the remaining mixture thus formed. If it is the case the corn fiber oil does not remain in the product mixture formed upon the hydrolysis of corn fiber, or if it is not desired to separate corn fiber oil, cellulose may be separated from the product mixture formed upon hydrolysis. The cellulose may be separated via mechanical techniques such as filtration, screening, or centrifugation.

Upon separation of cellulose, a hemicellulose-containing liquid will be provided. This liquid may be subjected to ultrafiltration or a like mechanical process to recover hemicellulose. The remaining mixture may be subjected to a process such as selective ion exchange chromatography to recover phenolic acids, such as ferulic acid. The remaining liquid may be disposed of or may be used in applications such as the preparations of an animal feed. If the fiber has not been subjected to treatment with an amylase or protease enzyme, the hemicellulose obtained from the hemicellulose-contain liquid will include starch, polysaccharides, and proteins. This hemicellulose may be subjected to treatment with an amylase, a protease, or both to yield a liquid from which purer hemicellulose may be recovered via ultrafiltration. Alternatively, it is contemplated that the starch, polysaccharides, and proteins may be separated via other means.

It is contemplated that the hydrolysis of corn fiber may be catalyzed with a hemicellulose ferulate esterase, a coumeric esterase, and an acetate esterase, or a combination of two or more of the forgoing enzymes. In a highly preferred embodiment, a hemicellulose ferulate esterase, a coumeric acid and an acetate esterase are employed to catalyze the hydrolysis of various esters bonds. In the most preferred embodiments of the invention, an amylase or protease enzyme is used concurrently in conjunction with all of the foregoing enzymes. In these embodiments, the temperature should be the optimum temperature for the enzyme employed, or, when multiple enzymes are employed, the temperature that is optimum for the combination of enzymes. It is believed that the optimum temperature in most cases will fall within the temperature range hereinbefore provided.

The invention further contemplates the hydrolysis of hemicellulose with an arabinose-releasing enzyme to yield a mixture of xylan and arabinose. Preferably, but not necessarily, the hemicellulose is that obtained in the ultrafiltration step hereinbefore discussed. The hemicellulose is hydrolyzed in the presence of a catalytic amount of arabinose-releasing enzyme, such as an arabinofuranosidase, to yield the mixture of xylan and arabinose. While a range of hydrolysis conditions may be employed, generally, for acid arabinofuranosidases, the pH should be in the range from about 3-6. If an alkaline enzyme is found, the pH should be in the range optimal for the enzyme. In either case, the temperature should be in the range optimal for catalytic activity. The dosage of the enzyme should be such as to render the reaction complete (as measured by arabinose release) or as complete as economically practical within 2-12 hours. The xylan and/or arabinose thus formed may be separated from the resulting product mixture to yield arabinose and/or xylan via techniques such as ultrafiltration, column chromatography, or solvent precipitation of xylan. In further processing steps, the xylan may be hydrolyzed in accordance with known methods to yield xylose. Optionally, the xylose may be oxidized to form a diacid or hydrogenated to form xylitol.

The following non-limiting examples are provided to illustrate the present invention.

### EXAMPLE 1

### Solubilization of Corn Hulls

Corn hulls from a corn wet milling process were ground in an Alpine Mill to have the following screen profile.

| Sieve Mesh | 30 | 60 | 80 | 100 | 120 | 140 | Pan |
|---|---|---|---|---|---|---|---|
| % on | 0.2 | 46.9 | 31.9 | 15.3 | 2.1 | 2.2 | 1.4 |

The ground hulls, 250.0 g dry basis, were added to 2500 g reverse-osmosis (RO) water (pH=6.08) in a 5 L RB resin kettle. The kettle was equipped with a heating mantle, a stirrer, and a water-cooled reflux condenser. The system was protected from atmospheric carbon dioxide by an Ascarite™ trap.

The system was temperature controlled at 70° C., and 0.5 M NaOH was added from a burette in order to achieve pH=9.0. The mixture consumed NaOH, and it was necessary to add additional 0.5 M NaOH intermittently in order to reachieve pH=9.0.

The following table conveys the pH of the reaction mixture over time and with the addition of 0.5 M NaOH in increments in order to maintain pH=9.00. For example, the entry ↓8.87 [symbol] 9.00, in the pH column conveys that the pH had fallen to 8.87 in the time since the preceding addition of 0.5 M NaOH to reachieve pH=9.00. Uptake rates of NaOH in grams per minute were calculated by dividing the mass of NaOH added by the time elapsed since the preceding pH adjustment to 9.00.

| | | Total | | | | Total | |
|---|---|---|---|---|---|---|---|
| | Total g | Elapsed | | | | Volume | |
| | NaOH | Time | | Δ Time | ′g NaOH/ | 0.5 M | % NaOH |
| ′pH | added | (Minutes) | Δ g NaOH | (Minutes) | Min | NaOH | on Hulls |
| 3.98 | 0 | 0 | 0 | 0 | | 0 mL | 0 |
| 4.06 | 0 | | 0 | 0 | | 0 mL | 0 |
| 5.0 | 0.80 | | | | | 40 mL | 0.32 |
| 6.0 | 1.34 | | | | | 67 mL | 0.54 |
| 7.0 | 1.80 | | | | | 90 mL | 0.72 |
| 7.25 | 2.00 | | | | | 100 mL | 0.80 |
| 8.00 | 2.64 | | | | | 132 mL | 1.06 |
| 8.48 | 4.00 | | | | | 200 mL | 1.60 |
| 9.00 | 5.20 | 40 | | | | 254 mL | 2.08 |
| ↓8.87 = 9.00 | 5.78 | 45 | .68 | 5 | .116 | 289 mL | 2.31 |
| ↓8.87 = 9.00 | 6.32 | 55 | .64 | 10 | .054 | 318 mL | 2.53 |
| ↓8.80 = 9.00 | 6.84 | 63 | .52 | 8 | .065 | 342 mL | 2.74 |
| ↓8.83 = 9.00 | 7.14 | 70 | .30 | 15 | .020 | 357 mL | 2.88 |
| ↓8.67 = 9.00 | 7.42 | 77 | .28 | 14 | .020 | 371 mL | 2.97 |
| ↓8.67 = 9.00 | 7.74 | 85 | .32 | 16 | .020 | 387 mL | 3.10 |
| ↓8.69 = 9.00 | 8.32 | 108 | .58 | 29 | .020 | 416 mL | 3.33 |
| ↓8.79 = 9.00 | 8.68 | 126 | .36 | 18 | .020 | 434 mL | 3.47 |
| ↓8.91 = 9.00 | 8.86 | 132 | .18 | 9 | .020 | 443 mL | 3.54 |
| ↓8.96 = 9.00 | 8.96 | 138 | .10 | 6 | .017 | 448 mL | 3.58 |
| ↓5.67 = 9.00 | 9.50 | 177 | .54 | 27 | .020 | 475 mL | 3.80 |
| ↓8.89 = 9.00 | 9.72 | 191 | .22 | 11 | .020 | 486 mL | 3.89 |
| ↓8.80 = 9.00 | 10.14 | 217 | .42 | 21 | .020 | 507 mL | 4.06 |
| ↓8.79 = 9.00 | 10.48 | 250 | .34 | 33 | .010 | 523 mL | 4.19 |
| ↓7.87 = 9.00 | 12.48 | 1120 | 2.00 | — | — | 624 mL | 4.96 |
| ↓8.90 = 9.00 | 12.68 | 1135 | .20 | 15 | .013 | 634 mL | 5.07 |
| ↓8.93 = 9.00 | 12.82 | 1150 | .14 | 15 | .009 | 641 mL | 5.13 |
| ↓8.89 = 9.00 | 13.06 | 1180 | .24 | 30 | .008 | 653 mL | 5.22 |
| ↓8.89 = 9.00 | 13.32 | 1210 | .26 | 30 | .009 | 666 mL | 5.33 |
| ↓8.93 = 9.00 | 13.46 | 1240 | .14 | 30 | .005 | 673 mL | 5.38 |
| ↓8.89 = 9.00 | 13.70 | 1270 | .24 | 30 | .008 | 685 mL | 5.48 |
| ↓8.95 = 9.00 | 13.82 | 1300 | .12 | 30 | .004 | 691 mL | 5.63 |
| ↓8.90 = 9.00 | 14.00 | 1330 | .18 | 30 | .006 | 700 mL | 5.60 |
| ↓8.92 = 9.00 | 14.18 | 1360 | .18 | 30 | .006 | 709 mL | 5.67 |
| ↓8.94 = 9.00 | 14.30 | 1390 | .12 | 30 | .004 | 715 mL | 5.72 |
| ↓8.89 = 9.00 | 14.58 | 1450 | .26 | 60 | .004 | 728 mL | 5.82 |
| ↓8.95 = 9.00 | 14.82 | 1570 | .25 | 120 | .002 | 741 mL | 5.93 |
| ↓8.85 = 9.00 | 15.20 | 1690 | .38 | 120 | .002 | 760 mL | 6.08 |
| ↓8.59 = 9.00 | 15.16 | 2580 | .95 | — | — | 808 mL | 5.45 |
| ↓8.97 = 9.00 | 16.24 | 2620 | .06 | 60 | .001 | 612 mL | 6.50 |
| ↓8.96 = 9.00 | 16.36 | 2880 | .12 | 60 | .002 | 818 mL | 6.54 |
| ↓5.98 = 9.00 | 16.42 | 2740 | .06 | 60 | .001 | 821 mL | 6.57 |
| ↓8.95 = 9.00 | 16.54 | 2800 | .12 | 60 | .002 | 827 mL | 5.52 |
| ↓8.98 = 9.00 | 16.62 | 2880 | .08 | 60 | .001 | 831 mL | 6.65 |
| ↓8.94 = 9.00 | 16.76 | 2980 | .14 | 120 | .001 | 838 mL | 6.70 |
| ↓8.92 = 9.00 | 16.96 | 3100 | .20 | 120 | .002 | 848 mL | 6.78 |
| ↓8.63 = 9.00 | 17.90 | 4000 | .94 | — | — | 895 mL | 7.96 |

### EXAMPLE 2

Corn hulls from a corn wet milling process are ground in Alpine Mill as in Example 1. The ground hulls, 250.0 g dry are added to 2500 g RO water (pH=6.08) in a 5 L RB resin kettle. The kettle is equipped with a heating mantle, stirrer, and a water-cooled reflux condenser. The system is protected from atmospheric carbon dioxide by an Ascarite™ trap.

The system is temperature controlled at 70° C., and 0.5 M NaOH is added from a burette in order to achieve pH=9.0. A catalyzing enzyme, hemicellulose ferulate esterase, is added to the kettle at least in sufficient amount (expressed as number of enzyme units) in order to complete the reaction within 2-12 hours, and the corn hulls are allowed to become hydrolyzed. Additional 0.5 M NaOH is intermittently or continuously added to maintain the pH in the range from 8.5 to 9.5.

### EXAMPLE 3

Example 2 is repeated, except that the catalyzing enzyme is coumeric acid esterase which is added in at least an amount effective to cease release of dicoumeric within 2-12 hours.

### EXAMPLE 4

Example 2 is repeated, except the catalyzing enzyme is acetate esterase which is dosed as in a manner similar to that heretofore discussed.

### EXAMPLE 5

Example 2 is repeated, except that two catalytic enzymes, hemicellulose ferulate esterase and coumeric acid esterase, are employed.

### EXAMPLE 6

Example 2 is repeated, except that two catalyzing enzymes, hemicellulose ferulate esterase and acetate esterase, are employed.

### EXAMPLE 7

Example 2 is repeated, except that two catalyzing enzymes, coumeric acid esterase and acetate esterase, are employed.

### EXAMPLE 8

Example 1 is repeated, except that hemicellulose ferulate esterase, coumeric acid esterase, and acetate esterase all are employed as catalyzing enzymes.

### EXAMPLE 9

Example 8 is repeated, except that an amylase enzyme and protease enzyme are also employed concurrently as catalyzing enzymes.

### EXAMPLE 10

The product mixture prepared in accordance with Example 9 is centrifuged or skimmed to recover corn fiber oil. The remaining mixture is filtered to recover cellulose. The remaining hemicellulose-containing liquid is ultrafiltered to recover hemicellulose.

### EXAMPLE 11

Hemicellulose, 10% solution in water, is brought to a pH of 3-6 (7-10 for an alkaline arabinofuranosidose) and an amount of enzyme calculated to complete arabinose release within 2-12 hours is added. The temperature is brought to a temperature within the range optimal for the enzyme, which in this case is 70° C. A hemicellulose containing liquid is provided.

### EXAMPLE 12

Dry hulls, water and enzymes are brought to a solid content of 50-80% dry solid basis (more preferably 60-70%). The pH is adjusted to 9.5 and the mixture is fed to an extruder equipped with a water jacket, a pH controller, and an alkaline feed system. A paste is extruded from the barrel end of the extractor. The paste is extracted with water in a batch or continuous process Thus, it is seen that the invention provides a method for the enzymatic catalysis of corn fiber. The invention further provides methods for obtaining products such as corn fiber oil, cellulose, and hemicellulose from a product mixture formed upon hydrolysis of corn fiber. It is further seen that the invention also provides a method for hydrolyzing corn fiber under relatively mild conditions of temperature and pH.

While particular embodiments in the invention have been shown, the invention is not limited thereto, but rather is delimited by the full scope of the appended claims. All references cited herein are hereby incorporated by reference in their entireties.

what is claimed is:

1. A method for treating a grain fiber to form a product mixture, comprising:
subjecting said fiber to alkaline hydrolysis in the presence of a hemicellulose ferulate esterase in an aqueous medium, said hemicellulose ferulate esterase being present in an amount effective to catalyze said alkaline hydrolysis and said alkaline hydrolysis being performed under conditions suitable to permit said hemicellulose ferulate esterase to catalyze said alkaline hydrolysis, the initial pH of said aqueous medium being alkaline but sufficiently low to permit said enzyme to have catalytic activity.

2. A method according to claim 1, said fiber being corn fiber.

3. A method according to claim 1, comprising maintaining the pH of said aqueous medium at a level approximating said initial pH.

4. A method according to claim 3, the initial pH of said aqueous medium being less than 10.5.

5. A method according to claim 3, the initial pH of said aqueous medium being less than 10.0.

6. A method according to claim 1, the initial pH of said aqueous medium being in the range from 8.0 to 9.5, the method comprising maintaining the pH of said aqueous medium at a level approximately in the range from 8.0 to 9.5.

7. A method according to claim 1, said alkaline hydrolysis being further conducted in the presence of a coumeric acid esterase, said coumeric acid esterase being active at said initial pH and being present in an amount sufficient to catalyze the hydrolysis of said grain fiber.

8. A method according to claim 1, said alkaline hydrolysis being further conducted in the presence of an acetate esterase, said acetate esterase being active at said initial pH and being present in an amount sufficient to catalyze the hydrolysis of acetate bonds present in said grain fiber.

9. A method according to claim 8, said alkaline hydrolysis being further conducted in the presence of a coumeric acid esterase, said coumeric acid esterase being active at said initial pH and being present in an amount sufficient to catalyze the hydrolysis of said grain fiber.

10. A method according to claim 1, said aqueous medium comprising a mixture of grain fiber and water, wherein grain fiber is present in an amount no greater than about 25% by total weight of water and grain fiber.

11. A method according to claim 1, said aqueous medium comprising an extrudable mixture of grain fiber and water.

12. A method according to claim 1, further comprising pretreating said grain fiber with an amylase and a protease to thereby hydrolyze respectively starch and protein in said grain fiber.

13. A method according to claim 1, said alkaline hydrolysis being conducted in the presence of an amount of amylase sufficient to hydrolyze starch present in said fiber and in the presence of an amount of protease sufficient to hydrolyze proteins in said fiber, the conditions of said alkaline hydrolysis being effective to permit said amylase and said protease to have catalytic activity.

14. The product mixture formed by the method of claim 1.

15. The product mixture formed by the method of claim 9.

16. A method for obtaining corn fiber oil, comprising:
providing a product mixture, said product mixture having been prepared by:
subjecting corn fiber to alkaline hydrolysis in the presence of a hemicellulose ferulate esterase in an aqueous medium, said hemicellulose ferulate esterase being present in an amount effective to catalyze said alkaline hydrolysis and said alkaline hydrolysis being performed under conditions suitable to permit said hemicellulose ferulate esterase to catalyze said alkaline hydrolysis, the initial pH of said aqueous medium being alkaline but sufficiently low to permit said enzyme to have catalytic activity; and
mechanically separating corn fiber oil from said product mixture.

17. A method according to claim 16, comprising maintaining the pH of said aqueous medium at a level approximating said initial pH.

18. A method according to claim 17, the initial pH of said aqueous medium being less than 10.5.

19. A method according to claim 17, the initial pH of said aqueous medium being less than 10.0.

20. A method according to claim 16, the initial pH of said aqueous medium being in the range from 8.0 to 9.5, the method comprising maintaining the pH of said aqueous medium at a level approximately in the range from 8.0 to 9.5.

21. A method according to claim 16, said alkaline hydrolysis being further conducted in the presence of a coumeric acid esterase, said coumeric acid esterase being active at said initial pH and being present in an amount sufficient to catalyze the hydrolysis of said corn fiber.

22. A method according to claim 16, said alkaline hydrolysis being further conducted in the presence of an acetate esterase, said acetate esterase being active at said initial pH and being present in an amount sufficient to catalyze the hydrolysis of acetate bonds present in said corn fiber.

23. A method according to claim 22, said alkaline hydrolysis being further conducted in the presence of a coumeric acid esterase, said coumeric acid esterase being active at said initial pH and being present in an amount sufficient to catalyze the hydrolysis of said corn fiber.

24. A method according to claim 16, said aqueous medium comprising a mixture of corn fiber and water wherein corn fiber is present in an amount no greater than about 25% by total weight of water and corn fiber.

25. A method according to claim 16, said aqueous medium comprising an extrudable mixture of corn fiber and water.

26. A method according to claim 16, further comprising pretreating said grain fiber with an amylase and a protease to thereby hydrolyze respectively starch and protein in said grain fiber.

27. A method according to claim 16, said alkaline hydrolysis being conducted in the presence of an amount of amylase sufficient to hydrolyze starch present in said fiber and in the presence of an amount of protease sufficient to hydrolyze proteins in said fiber, the conditions of said alkaline hydrolysis being effective to permit said amylase and said protease to have catalytic activity.

28. The corn fiber oil prepared in accordance with claim 16.

29. The corn fiber oil prepared in accordance with claim 23.

30. A method for obtaining a grain fiber cellulose, comprising:
providing a product mixture, said product mixture having been prepared by subjecting a grain fiber to alkaline hydrolysis in the presence of a hemicellulose ferulate esterase in an aqueous medium, said hemicellulose ferulate esterase being present in an amount effective to catalyze said alkaline hydrolysis and said alkaline hydrolysis being performed under conditions suitable to permit said hemicellulose ferulate esterase to catalyze said alkaline hydrolysis, the initial pH of said aqueous medium being alkaline but sufficiently low to permit said enzyme to have catalytic activity;
optionally mechanically separating grain fiber oil from said product mixture thereby leaving a remaining mixture; and
mechanically separating grain fiber cellulose from said remaining mixture.

31. A method according to claim 30, said fiber being corn fiber.

32. A method according to claim 30, comprising maintaining the pH of said aqueous medium at a level approximating said initial pH.

33. A method according to claim 32, the initial pH of said aqueous medium being less than 10.5.

34. A method according to claim 32, the initial pH of said aqueous medium being less than 10.0.

35. A method according to claim 30, the initial pH of said aqueous medium being in the range from 8.0 to 9.5, the method comprising maintaining the pH of said aqueous medium at a level approximately in the range from 8.0 to 9.5.

36. A method according to claim 30, said alkaline hydrolysis being further conducted in the presence of a coumeric acid esterase, said coumeric acid esterase being active at said initial pH and being present in an amount sufficient to catalyze the hydrolysis of said grain fiber.

37. A method according to claim 30, said alkaline hydrolysis being further conducted in the presence of an acetate esterase, said acetate esterase being active at said initial pH and being present in an amount sufficient to catalyze the hydrolysis of acetate bonds present in said grain fiber.

38. A method according to claim 37, said alkaline hydrolysis being further conducted in the presence of a coumeric acid esterase, said coumeric acid esterase being active at said initial pH and being present in an amount sufficient to catalyze the hydrolysis of said grain fiber.

39. A method according to claim 30, said aqueous medium comprising a mixture of grain fiber and water wherein grain fiber is present in an amount no greater than about 25% by total weight of water and grain fiber.

40. A method according to claim 30, said aqueous medium comprising an extrudable mixture of grain fiber and water.

41. A method according to claim 30, further comprising pretreating said grain fiber with an amylase and a protease to thereby hydrolyze respectively starch and protein in said grain fiber.

42. A method according to claim 30, said alkaline hydrolysis being conducted in the presence of an amount of amylase sufficient to hydrolyze starch present in said fiber and in the presence of an amount of protease sufficient to hydrolyze proteins in said fiber, the conditions of said alkaline hydrolysis being effective to permit said amylase and said protease to have catalytic activity.

43. The grain fiber cellulose product obtained by the method of claim 30.

44. The grain fiber cellulose product obtained by the method of claim 37.

45. A method for obtaining hemicellulose, comprising:
providing a product mixture, said product mixture having been prepared by subjecting a grain fiber to alkaline hydrolysis in the presence of a hemicellulose ferulate esterase in an aqueous medium, said hemicellulose ferulate esterase being present in an amount effective to catalyze said alkaline hydrolysis and said alkaline hydrolysis being performed under conditions suitable to permit said hemicellulose ferulate esterase to catalyze said alkaline hydrolysis, the initial pH of said aqueous medium being alkaline but sufficiently low to permit said enzyme to have catalytic activity;
optionally mechanically separating grain fiber oil from said product mixture thereby leaving a remaining mixture;
mechanically separating grain fiber cellulose from said remaining mixture thereby leaving a hemicellulose-containing liquid; and
mechanically separating hemicellulose from said hemicellulose-containing liquid.

46. A method according to claim 45, said grain fiber being corn fiber.

47. A method according to claim 45, comprising maintaining the pH of said aqueous medium at a level approximating said initial pH.

48. A method according to claim 47, the initial pH of said aqueous medium being less than 10.5.

49. A method according to claim 47, the initial pH of said aqueous medium being less than 10.0.

50. A method according to claim 45, the initial pH of said aqueous medium being in the range from 8.0 to 9.5, the method comprising maintaining the pH of said aqueous medium at a level approximately in the range from 8.5 to 9.5.

51. A method according to claim 45, said alkaline hydrolysis being further conducted in the presence of a coumeric acid esterase, said coumeric acid esterase being active at said initial pH and being present in an amount sufficient to catalyze the hydrolysis of said grain fiber.

52. A method according to claim 45, said alkaline hydrolysis being further conducted in the presence of an acetate esterase, said acetate esterase being active at said initial pH and being present in an amount sufficient to catalyze the hydrolysis of acetate bonds present in said grain fiber.

53. A method according to claim 52, said alkaline hydrolysis being further conducted in the presence of a coumeric acid esterase, said coumeric acid esterase being active at said initial pH and being present in an amount sufficient to catalyze the hydrolysis of said grain fiber.

54. A method according to claim 45, said aqueous medium comprising a mixture of grain fiber and water wherein grain fiber is present in an amount no greater than about 25% by total weight of water and grain fiber.

55. A method according to claim 45, said aqueous medium comprising an extrudable mixture of grain fiber and water.

56. A method according to claim 45, further comprising pretreating said grain fiber with an amylase and a protease to thereby hydrolyze respectively starch and protein in said grain fiber.

57. A method according to claim 45, said alkaline hydrolysis being conducted in the presence of an amount of amylase sufficient to hydrolyze starch present in said fiber and in the presence of an amount of protease sufficient to hydrolyze proteins in said fiber, the conditions of said alkaline hydrolysis being effective to permit said amylase and said protease to have catalytic activity.

58. The hemicellulose product obtained by the method of claim 45.

59. The hemicellulose product obtained by the method of claim 53.

60. A method for obtaining a mixture of arabinose and xylan, comprising;
providing a product mixture, said product mixture having been prepared by subjecting a grain fiber to alkaline hydrolysis in the presence of a hemicellulose ferulate esterase in an aqueous medium, said hemicellulose ferulate esterase being present in an amount effective to catalyze said alkaline hydrolysis and said alkaline hydrolysis being performed under conditions suitable to permit said hemicellulose ferulate esterase to catalyze said alkaline hydrolysis, the initial pH of said aqueous medium being alkaline but sufficiently low to permit said enzyme to have catalytic activity;
optionally mechanically separating grain fiber oil from said product mixture thereby leaving a remaining mixture;
mechanically separating grain fiber cellulose from said remaining mixture thereby leaving a hemicellulose-containing liquid:
mechanically separating hemicellulose from said hemicellulose-containing liquid; and
treating the hemicellulose thus formed in an aqueous solution with an arabinose-releasing enzyme under reaction conditions suitable to yield a mixture of arabinose and xylan.

61. A xylan product prepared by separating xylan from the mixture formed in accordance with claim 60.

62. A xylose product prepared by hydrolyzing the xylan of claim 61.

63. A method for obtaining a mixture of arabinose and xylan, comprising;
providing an aqueous solution of hemicellulose; and
treating said hemicellulose in said aqueous solution with an arabinose-releasing enzyme under reaction conditions suitable to yield a mixture of arabinose and xylan.

64. The xylan product prepared by separating xylan from the mixture formed in accordance with claim 63.

65. A xylose product prepared by hydrolyzing the xylan of claim 64.
